(19) United States
(12) Patent Application Publication
(10) Pub. No.: US 2025/0191745 A1
(43) Pub. Date: Jun. 12, 2025

(54) HARDWARE IDENTITY AUTHENTICATION METHOD FOR MEDICAL DEVICE AND MEDICAL DEVICE

(71) Applicant: GE Precision Healthcare LLC, Waukesha, WI (US)

(72) Inventors: Youli Sun, Wuxi (CN); Zhihua Jia, Wuxi (CN); Weijie Zhang, Wuxi (CN); Guoping Li, Wuxi (CN); Weiming Du, Wuxi (CN)

(21) Appl. No.: 18/977,420

(22) Filed: Dec. 11, 2024

(30) Foreign Application Priority Data
Dec. 11, 2023 (CN) 202311693205.8

(51) Int. Cl. G16H 40/40; G06F 21/44; H04L 9/08

(52) U.S. Cl. CPC G16H 40/40; G06F 21/44; H04L 9/0819; H04L 9/0866

(57) ABSTRACT

The hardware identity authentication method for a medical device includes, after all hardware to be authenticated of the medical device is started, acquiring unique identifiers of all the hardware to be authenticated; calculating a first key according to the unique identifiers of all the hardware to be authenticated; and matching the first key with a pre-stored second key, and determining whether the identity authentication is passed according to a matching result. All the hardware to be authenticated is bound together for authentication, which can effectively prevent replacement of an entire module, so that hardware maintenance of the medical device may be performed only in an authorized state, thereby improving the reliability of a system and the quality of hardware maintenance.

### CROSS-REFERENCE TO RELATED APPLICATION

This application claim priority to Chinese Patent Application No. 202311693205.8, which was file on Dec. 11, 2023 at the Chinese Patent Office. The entire contents of the above-listed application are incorporated by reference herein in their entirety.

### TECHNICAL FIELD

Embodiments of the present application relate to the technical field of medical devices, and in particular relate to a hardware identity authentication method for a medical device, and a medical device

### BACKGROUND

Medical devices, also referred to as medical treatment devices, include diagnostic devices, therapeutic devices, auxiliary devices and the like, and medical devices represented by ultrasound devices play an important role in the field of ultrasound.

Ultrasound devices can help physicians perform a large number of examination tasks. Thus, the health status of the ultrasound device is very important, the most important of which is the hardware status. When an abnormality occurs, the ultrasound device not only cannot work properly, but may even cause injury to an associated person.

Currently, although a simple hardware protection mechanism is available for the hardware of the ultrasound device, the core components thereof cannot be covered. A person with a certain skill basis can easily complete hardware replacement while circumventing this protection mechanism. The easy replacement of hardware without authorization may cause the quality of the medical device to be unreliable, and there will be security risks for both users and patients.

It should be noted that the above introduction of the background is only for the convenience of clearly and completely describing the technical solutions of the present application, and for the convenience of understanding for those skilled in the art.

### SUMMARY

In view of the above problems or other similar problems pointed out in the background, embodiments of the present application provide a hardware identity authentication method for a medical device and a medical device, so as to effectively prevent unauthorized maintenance by means of a hardware identity verification chain.

According to one aspect of the embodiments of the present application, a hardware identity authentication method for a medical device is provided. The method comprises:
After all hardware to be authenticated of the medical device is started, acquiring unique identifiers of all the hardware to be authenticated;
Calculating a first key according to the unique identifiers of all the hardware to be authenticated; and
Matching the first key with a pre-stored second key, and determining whether the identity authentication is passed according to a matching result.

According to another aspect of the embodiments of the present application, a medical device is provided. The medical device comprises:
At least one first component, each of the first components storing a unique ID of said first component;
A first OTP-ROM, the first OTP-ROM storing a unique ID of the first OTP-ROM, and storing a second key;
At least one second component, each second component comprising a second OTP-ROM, and the second OTP-ROM of each second component storing a unique ID of said second OTP-ROM; and
A processor connected to the first component, the first OTP-ROM and the second component, and configured to perform the foregoing hardware identity authentication method for a medical device.

According to yet another aspect of the embodiments of the present application, a non-transitory computer-readable medium is provided. The non-transitory computer-readable medium has a computer program stored therein, the computer program having at least one code segment, and the at least one code segment being executable by a machine so as to cause the machine to perform the steps of the foregoing hardware identity authentication method for a medical device.

One of the beneficial effects of the embodiments of the present application is that: according to the embodiments of the present application, all the hardware to be authenticated is bound together for authentication, which can effectively prevent replacement of an entire module, so that hardware maintenance of the medical device may be performed only in an authorized state, thereby improving the reliability of a system and the quality of hardware maintenance.

With reference to the following description and drawings, specific implementations of the embodiments of the present application are disclosed in detail, and the means by which the principles of the embodiments of the present application can be employed are illustrated. It should be understood that the embodiments of the present application are not limited in scope thereby. Within the scope of the spirit and clauses of the appended claims, the embodiments of the present application include many changes, modifications, and equivalents.

### BRIEF DESCRIPTION OF THE DRAWINGS

The included drawings are used to provide further understanding of the embodiments of the present application, which constitute a part of the description and are used to illustrate the implementations of the present application and explain the principles of the present application together with textual description. Evidently, the drawings in the following description are merely some embodiments of the present application, and a person of ordinary skill in the art may obtain other implementations according to the drawings without involving inventive effort. In the drawings:

FIG. 1 is a schematic diagram of a hardware identity authentication method for a medical device according to an embodiment of the present application;

FIG. 2 is a schematic diagram of a hardware structure of an ultrasound device;

FIG. 3 is a schematic diagram of a startup process of the ultrasound device based on the hardware structure of the ultrasound device shown in FIG. 2;

FIG. 4 is a schematic diagram of a generation process of a key;

FIG. 5 is a schematic diagram of a user interface of an external computer PC;

FIG. 6 is a schematic diagram of an interaction between an external computer and a medical device;

FIG. 7 is a schematic diagram of a process of generating and configuring a second key by an external computer; and

FIG. 8 is a schematic diagram of a hardware structure of a medical device according to an embodiment of the present application.

### DETAILED DESCRIPTION

The foregoing and other features of the embodiments of the present application will become apparent from the following description with reference to the drawings. In the description and drawings, specific implementations of the present application are disclosed in detail, and part of the implementations in which the principles of the embodiments of the present application may be employed are indicated. It should be understood that the present application is not limited to the described implementations. On the contrary, the embodiments of the present application include all modifications, variations, and equivalents which fall within the scope of the appended claims.

In the embodiments of the present application, the terms “first”, “second”, etc., are used to distinguish different elements by their title, but do not represent a spatial arrangement or temporal order, etc., of these elements, and these elements should not be limited by these terms. The term “and/or” includes any and all combinations of one or more associated listed terms. The terms “comprise”, “include”, “have”, etc., refer to the presence of described features, elements, components, or assemblies, but do not exclude the presence or addition of one or more other features, elements, components, or assemblies.

In the embodiments of the present application, the singular forms “a” and “the”, etc., include plural forms, and should be broadly construed as “a type of” or “a class of” rather than being limited to the meaning of “one”. Furthermore, the term “the” should be construed as including both the singular and plural forms, unless otherwise specified in the context. In addition, the term “according to” should be construed as “at least in part according to . . . ” and the term “on the basis of” should be construed as “at least in part on the basis of . . . ”, unless otherwise specified in the context.

The features described and/or illustrated for one embodiment may be used in one or more other embodiments in an identical or similar manner, combined with features in other embodiments, or replace features in other embodiments. The term “include/comprise” when used herein refers to the presence of features, integrated components, steps, or assemblies, but does not exclude the presence or addition of one or more other features, integrated components, steps, or assemblies.

Embodiments of the present application provide a hardware identity authentication method for a medical device. The medical device may include, for example, at least one of an ultrasound device, a monitor, an electrocardiogram anesthesia machine, a ventilator, an X-ray imaging device, and a magnetic resonance imaging device. The X-ray imaging device may include, for example, a medical device that performs imaging using X-rays, such as an X-ray device, a digital radiography (DR) device, and a computed tomography (CT) device.

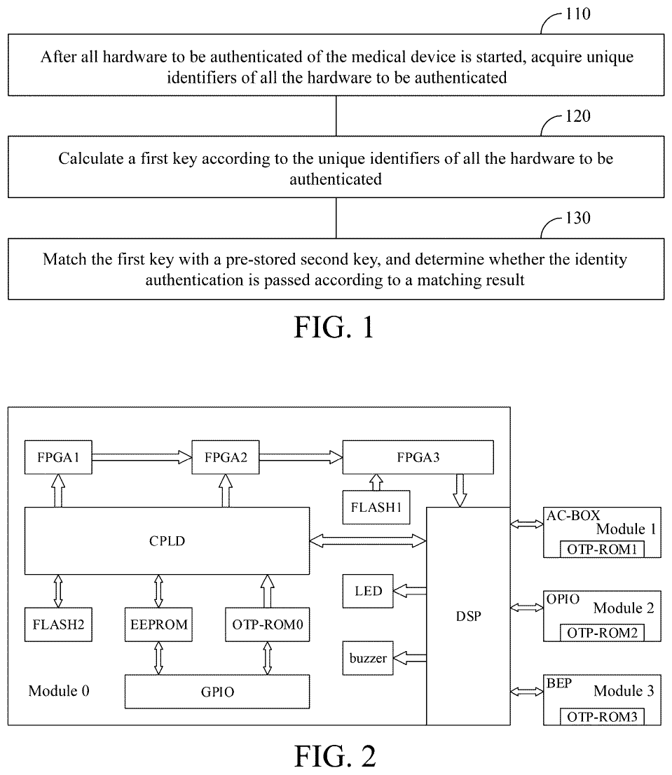

FIG. 1 is a schematic diagram of a hardware identity authentication method for a medical device according to an embodiment of the present application. As shown in FIG. 1, the method includes:
110: After all hardware to be authenticated of the medical device is started, acquiring unique identifiers of all the hardware to be authenticated;
120: Calculating a first key according to the unique identifiers of all the hardware to be authenticated; and
130: Matching the first key with a pre-stored second key, and determining whether the identity authentication is passed according to a matching result.

It should be noted that FIG. 1 merely schematically illustrates the embodiment of the present application, but the present application is not limited thereto. For example, the order of execution between operations may be appropriately adjusted. In addition, some other operations may be added or some operations may be omitted. Those skilled in the art may make appropriate variations according to the above content, rather than being limited to the disclosure of FIG. 1.

According to the above embodiment, the first key obtained based on the unique identifiers of all the hardware to be authenticated is matched with the pre-stored second key, and whether the hardware to be authenticated passes the identity authentication is determined according to the matching result. As such, all the hardware to be authenticated is bound together for authentication, which can effectively prevent replacement of an entire module, so that hardware maintenance of the medical device may be performed only in an authorized state, thereby improving the reliability of a system and the quality of hardware maintenance.

In the above embodiment, all the hardware to be authenticated may be all hardware of the medical device, or may be a part of critical hardware of the medical device, e.g., hardware requiring maintenance, hardware having a high likelihood of being illegally replaced, etc.

In some embodiments, the hardware to be authenticated includes, for example, a processor, a first component, a one time programmable read-only memory) (referred to as a first OTP-ROM), a second component including an OTP-ROM (referred to as a second OTP-ROM), etc.

The processor is, for example, a digital signal processor (DSP) and has a unique identifier (or ID). For example, for the DSP, the unique ID thereof may be a media access control ID (MAC ID). The present application is not limited thereto. According to differences in medical devices, the processor may be another processor, such as a central processing unit (CPU) or a microprocessor unit (MPU). Accordingly, different types of processors may have different unique IDs.

In the above embodiment, the acquiring unique identifiers of the hardware to be authenticated is, for example: acquiring a unique identifier (e.g., MAC ID) of the processor. As such, the unique identifier of the processor is involved in a process of generating the first key, which prevents the processor from being illegally repaired or replaced, thereby improving the overall performance of the medical device.

The first component is, for example, a Field Programmable Gate Array (FPGA) or another logic device, and for convenience of description, the general term “first component” is used herein. The first component has a unique identifier. It should be noted that different first components may have different unique identifiers, and the name of the unique identifier of the first component is not limited in the present application. The first component is connected to the processor in series and/or in parallel.

In the above embodiment, there may be a plurality of first components, e.g., the number of first components may be two or three or more, and at least one of the first components is connected to a flash memory (referred to as a first flash memory) in which a startup program for self-starting the first component is stored. As such, upon power-up of the medical device, the first component storing the startup program may self-start without waiting for configuration of the processor.

In the above embodiment, acquiring a unique identifier of the hardware to be authenticated is, for example: acquiring a DNA of the FPGA. As such, the unique ID of the FPGA is involved in the process of generating the first key, which prevents the FPGA from being illegally repaired or replaced, thereby improving the overall performance of the medical device.

The first OTP-ROM is connected to the processor, and may be directly connected to the processor, and may also be connected to the processor via another component (referred to as a third component) such as a Complex Programmable Logic Device (CPLD). The first OTP-ROM may likewise have a unique identifier which alternatively may be referred to as a unique ID.

In the above embodiment, acquiring a unique identifier of the hardware to be authenticated is, for example: acquiring a unique ID of the first OTP-ROM. As such, the unique ID of the first OTP-ROM is involved in the process of generating the first key, which prevents a main board of the medical device from being illegally repaired or replaced, thereby improving the overall performance of the medical device.

The second component is, for example, an external apparatus, device or board, such as a power module, an operation input output module or a back end process module, and is connected to the processor in a wired or wireless manner; for convenience of description, the general term “second component” is used herein.

In the above embodiment, the number of second components may be one or more, each second component including an OTP-ROM (the second OTP-ROM), and the second OTP-ROM may also have a unique identifier, which alternatively may be a unique ID.

In the above embodiment, acquiring a unique identifier of the hardware to be authenticated is, for example: acquiring a unique ID of the second OTP-ROM. As such, the unique ID of the second OTP-ROM is involved in the process of generating the first key, which prevents the second component from being illegally repaired or replaced, thereby improving the overall performance of the medical device.

In the above embodiment, the pre-stored second key is associated with all the hardware to be authenticated, and the manner of generating the second key is not limited in the present application, for example, the second key may be obtained by acquiring in advance unique identifiers of the hardware to be authenticated, and then performing calculation based on the unique identifiers using a predetermined rule or algorithm, or the like.

In some embodiments, if the first key matches with the second key, it is determined that the hardware identity authentication of the medical device is passed, and if the first key does not match with the second key, it is determined that the hardware identity authentication of the medical device is not passed.

In the above embodiment, if the hardware identity authentication of the medical device is passed, that is, the first key matches with the second key, matching success prompting can be performed, for example, a message indicating that the authentication is passed is displayed on a display screen; for another example, matching success prompting is performed by blinking or illumination of a light emitting diode, and the like; and for another example, prompting that the authentication is passed is performed by emitting a sound of a predetermined frequency by a buzzer, or the like. Alternatively, if the hardware identity authentication of the medical device is passed, it is also possible to directly enter an operating system of the medical device to cause the medical device to be normally started without performing the matching success prompting.

In the above embodiment, if the hardware identity authentication of the medical device is not passed, that is, the first key does not match with the second key, matching failure prompting can be performed, for example, a message indicating that the authentication is not passed is displayed on a display screen; for another example, matching failure prompting is performed by blinking or illumination of a light emitting diode, and the like; and for another example, prompting that the authentication is not passed is performed by emitting a sound of a predetermined frequency by a buzzer, or the like.

In the above embodiment, if the hardware identity authentication of the medical device is not passed, the unique identifiers of the hardware to be authenticated and the first key may be further written into the memory, so as to facilitate workers locating a fault, to clarify which hardware has had an abnormal replacement or maintenance.

The hardware identity authentication method for a medical device according to an embodiment of the present application is described below using an ultrasound device as an example.

FIG. 2 is a schematic diagram of a hardware structure of an ultrasound device. As shown in FIG. 2, the ultrasound device includes four modules, i.e., module 0 to module 3.

The module 0 is an ultrasound board, and includes a DSP, an FPGA1 to an FPGA3, and an OTP-ROM0. The DSP functions as the described processor and has a unique identifier, each of the FPGA1 to the FPGA3 functions as a described first component and similarly has a unique identifier, and the OTP-ROM0 functions as the described first OTP-ROM and also has a unique identifier. The module 1 is an AC-BOX and functions as the described power module, the module 2 functions as the described user input module (an operation input output, or OPIO for short), and the module 3 functions as the described back end process module (BEP for short); the AC-BOX, the OPIO, and the BEP each include an OTP-ROM, namely, an OTP-ROM1 to an OTP-ROM3, and the OTP-ROM1 to the OTP-ROM3 each have a unique identifier.

In the above example, as shown in FIG. 2, the module 0 further includes a FLASH1 that is connected to the FPGA3 and stores a startup program for self-starting the FPGA3; a CPLD connected to the DSP, the FPGA1, the FPGA2 and the OTP-ROM0, a FLASH2 and an Electrically Erasable Programmable Read Only Memory (EEPROM) connected to the CPLD, a Light Emitting Diode (LED) and a buzzer connected to the DSP, and a General Purpose Input/Output Port (GPIO) connected to the EEPROM and the OTP-ROM0.

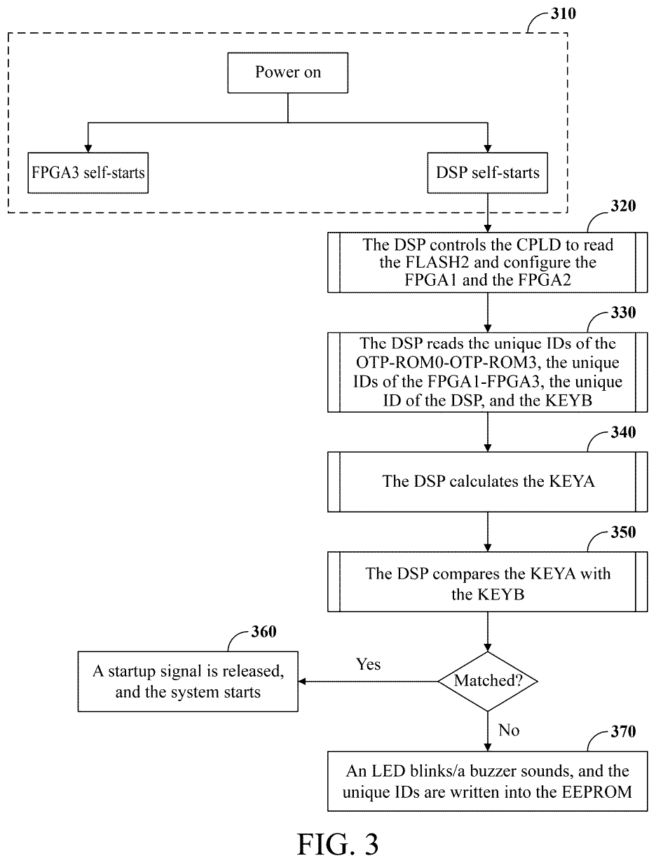

FIG. 3 is a schematic diagram of a startup process of the ultrasound device based on the hardware structure of the ultrasound device shown in FIG. 2.

As shown in FIG. 2 and FIG. 3, the startup process includes:
310: After the ultrasound device is powered on and starts a self-test, the FPGA3 and the DSP each automatically load corresponding startup programs, to complete self-starting;
320: The DSP controls the CPLD to perform the following operations: A) reading a minimum system program (i.e., startup program) of the FPGA in the FLASH2; and B) configuring the FPGA1 and the FPGA2 using the minimum system program;
330: The DSP reads the unique IDs of the OTP-ROM0 to the OTP-ROM3, the DNAs of the FPGA1-FPGA3, the unique ID of the DSP, and the second key (KEYB);

For example, the DSP reads the KEYB and the unique ID of the OTP-ROM0 from the OTP-ROM0, and reads the unique IDs of the OTP-ROM1 to the OTP-ROM3 from the OTP-ROM1 to the OTP-ROM3;

For another example, the FPGA1 sends the DNA thereof to the FPGA2, the FPGA2 packages and sends its own DNA and the DNA of the FPGA1 to the FPGA3, and finally the FPGA3 packages and sends all the DNAs to the DSP;

For another example, the DSP reads the unique ID of the DSP from a DSP chip;
340: The DSP calculates the first key (KEYA);

For example, the DSP calculates the KEYA using a specific algorithm according to the unique IDs of the FPGA1-FPGA3, the DSP, and the OTP-ROM0 to the OTP-ROM3;
350: The DSP compares the KEYA with the KEYB, and if the KEYA and the KEYB are equal, it indicates that the self-test is passed, and the process proceeds to 360; if the KEYA and the KEYB are not equal, it indicates that the self-test has failed, and the process proceeds to 370;
360: The DSP releases a startup signal, and the system starts normally;
370: The DSP writes all the unique IDs and the KEYB into the EEPROM and causes the LED to blink while causing the buzzer to sound.

In operation 330, the DSP may further read module IDs of the OTP-ROM1 to the OTP-ROM3 and use the module IDs as a basis for calculating the KEYA, thereby further enhancing the binding relationship between the external device/apparatus/board (second component) and the ultrasound board, and improving the quality of hardware maintenance and the reliability of the system.

In the embodiment of the present application, the algorithm for calculating the KEYA is not limited as long as a key associated with these unique identifiers can be calculated based on the unique identifiers of the hardware to be authenticated; for example, an Advanced Encryption Standard (AES) encryption algorithm, a Data Encryption Standard (DES) encryption algorithm, a Triple Data Encryption Algorithm (3DES), or the like may be used to calculate the KEYA.

In the embodiment of the present application, the unique identifiers of the FPGA and the DSP may be read by means of a Joint Test Action Group (JTAG), and reference may be specifically made to related technologies of the JTAG, which will not be described here. The present application is not limited thereto, and the DNA of the FPGA and the unique identifier of the DSP may be read by other methods.

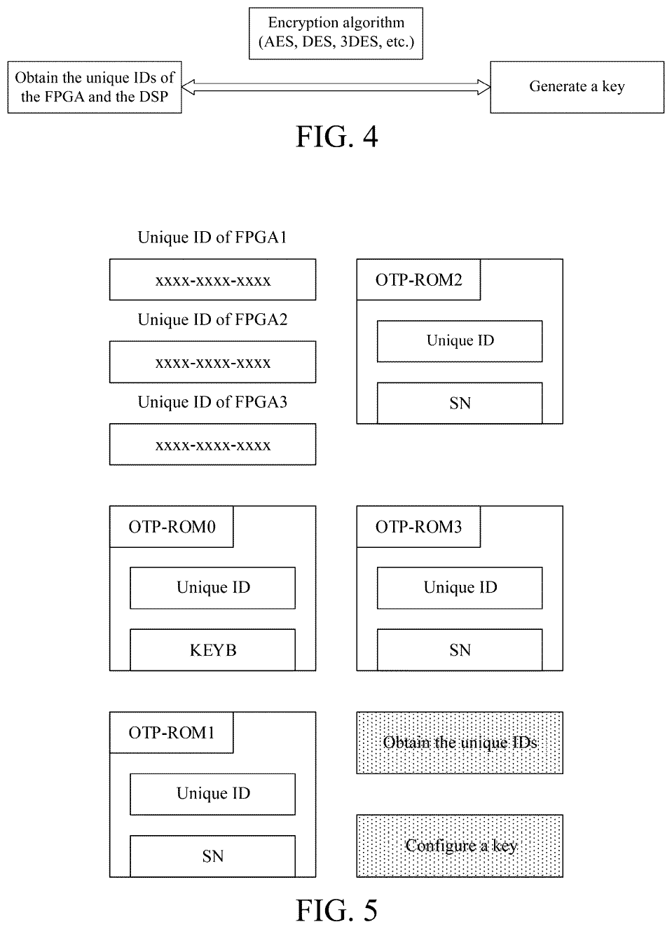

FIG. 4 is a schematic diagram of a generation process of a key. As shown in FIG. 4, the unique identifiers of the FPGA and the DSP are first read by means of a JTAG, each set of unique identifiers constituting a serial number; an encryption algorithm is then used to generate a key based on the serial number. As shown in FIG. 4, different keys are generated for different serial numbers. The encryption algorithm may be one or more of any existing algorithms, for example, may be one or more of an AES algorithm, a DES algorithm, and a 3DES algorithm. The present application does not make a limitation.

In the example of FIG. 4, the generation process of the key is briefly described, and the foregoing first key KEYA and second key KEYB can be obtained by the method shown in FIG. 4. The first key may be calculated by the DSP by acquiring the unique identifiers of the hardware to be authenticated when the medical device is powered on, as described above. The second key may be calculated by an external computer during a production process of the medical device by acquiring the unique identifier of the hardware to be authenticated, and a generation process of the second key is briefly described below.

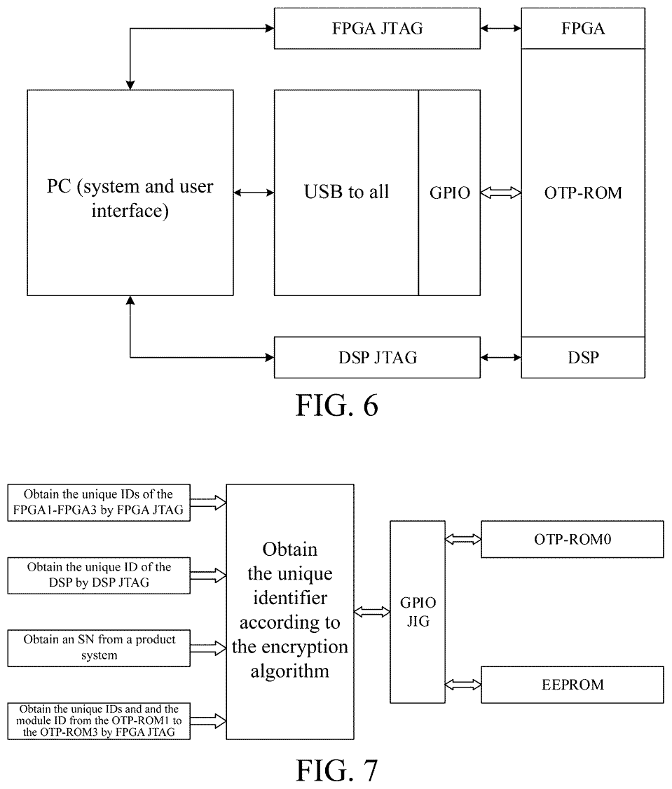

FIG. 5 is a schematic diagram of a user interface of an external computer PC, FIG. 6 is a schematic diagram of an interaction between an external computer and a medical device, and FIG. 7 is a schematic diagram of a process of generating and configuring a second key by an external computer.

As shown in FIG. 5, on the user interface, the FPGA1-FPGA3 and the OTP-ROM0 to the OTP-ROM3 are shown, the OTP-ROM0 stores the second key KEYB and a unique ID of the OTP-ROM0, and the OTP-ROM1 to the OTP-ROM3 store respective unique IDs and SNs (module IDs). In addition, on the user interface shown in FIG. 5, an acquisition button “GET ID” and a configuration button “CONFIG KEY” are also shown; the user can obtain the unique IDs of the FPGA1-FPGA3 and the OTP-ROM0 to the OTP-ROM3 by touching or pressing the acquisition button, and the user can configure the generated second key in the OTP-ROM0 by touching or pressing the configuration button.

As shown in FIG. 6 and FIG. 7, an external computer PC obtains the unique IDs of the FPGA1-FPGA3 from the FPGA1-FPGA3 by means of an FPGA JTAG, obtains the unique identifier of the DSP from the DSP by means of an DSP JTAG, obtains a SN of the medical device from a production task management system, and obtains the unique IDs and module IDs (optional) of the OTP-ROM1 to the OTP-ROM3 from the OTP-ROM1 to the OTP-ROM3; then, the unique identifiers are obtained according to the encryption algorithm, and the second key KEYB is generated based on the unique identifiers and stored in the OTP-ROM0 by means of a GPIO JIG, and in addition, the external computer PC may further store the second key KEYB in the EEPROM after generating the second key KEYB, thereby enhancing compatibility.

Although the generation of the second key KEYB has been described above with reference to FIGS. 5 to 7, the present application is not limited thereto, and the second key KEYB may be generated in other manners.

The above embodiments merely provide illustrative descriptions of the embodiments of the present application. However, the present application is not limited thereto, and appropriate variations may be made on the basis of the above embodiments. For example, each of the above embodiments may be used independently, or one or more among the above embodiments may be combined.

According to an embodiment of the present application, when the second key KEYB is generated, the unique ID in the OTP-ROM0 is bound, so that the generated second key KEYB is unique, and duplication and chip replacement of the second key KEYB can be effectively prevented. Also, since all the hardware to be authenticated is bound together for verification, replacement of the entire module can be effectively prevented. In addition, if the authentication fails due to illegal maintenance, illegal records can be effectively stored, which facilitates extraction and maintenance by legally authorized persons. In addition, according to the maintenance records, the Mean Time Between Failure (MTBF) of the module and the chip can be relatively accurately evaluated, which facilitates optimized design in research and development.

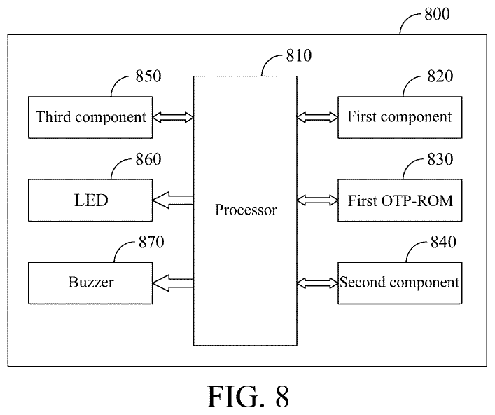

Embodiments of the present application further provide a medical device. FIG. 8 is a schematic diagram of a hardware structure of the medical device according to an embodiment of the present application. As shown in FIG. 8, the medical device 800 includes: a processor 810, as well as at least one first component 820, a first OTP-ROM 830 and at least one second component 840 connected to the processor 810.

In the above embodiment, each first component 820 stores a unique ID of said first component 820. In some embodiments, the first component 820 is, for example, the described FPGA. A description of the relevant content of the first component 820 has been made above, the contents of which are incorporated herein, and will not be described again here.

In the above embodiment, the first OTP-ROM 830 stores a unique ID of the first OTP-ROM 830 and a second key. In some embodiments, the first OTP-ROM 830 is, for example, the described OTP-ROM0. A description of the relevant content of the first OTP-ROM 830 has been made above, the contents of which are incorporated herein, and will not be described again here.

In the above embodiment, each second component 840 includes a second OTP-ROM in which a unique ID of said second OTP-ROM is stored. In some embodiments, the second component 840 is, for example, the described power module/operation input output module/back end process module, and the second OTP-ROM is, for example, the foregoing OTP-ROM1 to OTP-ROM3. In the foregoing embodiments, a description of the second component 840 and the second OTP-ROM has been made, the contents of which are incorporated herein, and will not be described again here.

In the above embodiment, the processor 810 is configured to perform the method described in the described embodiments. A description of the method has been made in the foregoing embodiment, the contents of which are incorporated herein and will not be described again here.

In some embodiments, as shown in FIG. 8, the medical device 800 further includes a third component 850 that is connected to the processor 810 and a flash memory (referred to as a second flash memory). A startup program is read from the second flash memory under the control of the processor 810, and the startup program is used to configure the at least one first component 820 to start the at least one first component 820.

In the above embodiment, the third component 850 is, for example, the CPLD of the described embodiment, and the at least one first component 820 is, for example, the FPGA1 and the FPGA2 of the described embodiment. A description of the third component 850 has been made in the foregoing embodiment, the contents of which are incorporated herein and will not be described again here.

In some embodiments, as shown in FIG. 8, the medical device 800 further includes a light emitting diode 860 and/or a buzzer 870, which are connected to the processor 810 for performing matching success and/or matching failure prompting under the control of the processor 810. A description of the prompting manner of the LED 860 and the buzzer 870 has been made in the foregoing embodiment, the contents of which are incorporated herein and will not be described again here.

In an embodiment of the present application, the medical device 800 may be the ultrasound device of the described embodiments; the present application is not limited thereto, and the medical device 800 may also be a monitor, an electrocardiogram anesthesia machine, a ventilator, an X-ray imaging device, a magnetic resonance imaging device, or the like.

The above embodiments only describe the structure and/or function of the medical device related to the present application, and the medical device further includes other structures and functions; see the related art for specifics.

In addition, the above embodiments merely provide illustrative descriptions of the embodiments of the present application. However, the present application is not limited thereto, and appropriate variations may be made on the basis of the above embodiments. For example, each of the above embodiments may be used independently, or one or more among the above embodiments may be combined.

According to the embodiments of the present application, all the hardware to be authenticated is bound together for authentication, which can effectively prevent replacement of an entire module, so that hardware maintenance of the medical device may be performed only in an authorized state, thereby improving the reliability of a system and the quality of hardware maintenance.

The embodiments of the present application further provide a non-transitory computer-readable medium having a computer program stored therein, the computer program having at least one code segment, and the at least one code segment being executable by a machine so as to cause the machine to perform steps of the method described in the foregoing embodiments.

The above apparatus and method of the present application can be implemented by hardware, or can be implemented by hardware in combination with software. The present application relates to such a computer-readable program that when executed by a logic component, the program causes the logic component to implement the foregoing apparatus or a constituent component, or causes the logic component to implement various methods or steps as described above. The present application further relates to a storage medium for storing the above program, such as a hard disk, a disk, an optical disk, a DVD, a flash memory, etc.

The method/apparatus described in view of the embodiments of the present application may be directly embodied as hardware, a software module executed by a processor, or a combination of the two. For example, one or more of the functional block diagrams and/or one or more combinations of the functional block diagrams shown in the drawings may correspond to either respective software modules or respective hardware modules of a computer program flow. The foregoing software modules may respectively correspond to the steps shown in the figures. The foregoing hardware modules can be implemented, for example, by firming the software modules using a field-programmable gate array (FPGA).

The software modules may be located in a RAM, a flash memory, a ROM, an EPROM, an EEPROM, a register, a hard disk, a portable storage disk, a CD-ROM, or any other form of storage medium known in the art. The storage medium may be coupled to a processor, so that the processor can read information from the storage medium and can write information into the storage medium. Alternatively, the storage medium may be a constituent component of the processor. The processor and the storage medium may be located in an ASIC. The software module may be stored in a memory of a mobile terminal, and may also be stored in a memory card that can be inserted into a mobile terminal. For example, if a device (such as a mobile terminal) uses a large-capacity MEGA-SIM card or a large-capacity flash memory device, the software modules can be stored in the MEGA-SIM card or the large-capacity flash memory apparatus.

One or more of the functional blocks and/or one or more combinations of the functional blocks shown in the accompanying drawings may be implemented as a general-purpose processor, a digital signal processor (DSP), an application-specific integrated circuit (ASIC), a field-programmable gate array (FPGA) or other programmable logic devices, discrete gate or transistor logic devices, a discrete hardware assembly, or any appropriate combination thereof for implementing the functions described in the present application. The one or more functional blocks and/or the one or more combinations of the functional blocks shown in the accompanying drawings may also be implemented as a combination of computing devices, such as a combination of a DSP and a microprocessor, multiple microprocessors, one or more microprocessors in communication combination with a DSP, or any other such configuration.

The present application is described above with reference to specific embodiments. However, it should be clear to those skilled in the art that the foregoing description is merely illustrative and is not intended to limit the scope of protection of the present application. Various variations and modifications may be made by those skilled in the art according to the spirit and principle of the present application, and these variations and modifications also fall within the scope of the present application.

Preferred embodiments of the present application are described above with reference to the accompanying drawings. Many features and advantages of the implementations are clear according to the detailed description, and therefore the appended claims are intended to cover all these features and advantages that fall within the true spirit and scope of these implementations. In addition, as many modifications and changes could be easily conceived of by those skilled in the art, the embodiments of the present application are not limited to the illustrated and described precise structures and operations, but can encompass all appropriate modifications, changes, and equivalents that fall within the scope of the implementations.

1. A hardware identity authentication method for a medical device, the method comprising:
after all hardware to be authenticated of the medical device is powered on, acquiring unique identifiers of all the hardware to be authenticated;
calculating a first key according to the unique identifiers of all the hardware to be authenticated; and
matching the first key with a pre-stored second key, and determining whether the identity authentication is passed according to the matching result.

2. The method according to claim 1, wherein determining whether the identity authentication is passed according to a matching result comprises:
based on the first key matching the second key, determining that the hardware identity authentication of the medical device is passed; and/or
based on the first key not matching the second key, determining that the hardware identity authentication of the medical device is not passed.

3. The method according to claim 1, wherein all the hardware to be authenticated comprises:
a processor;
a first component;
a first one time programmable read-only memory (OTP-ROM) in which the second key is stored; and
a second component including a second OTP-ROM, and
wherein acquiring unique identifiers of all the hardware to be authenticated comprises:
acquiring a unique ID of the processor;
acquiring a unique ID of the first component; and
acquiring unique IDs of the first OTP-ROM and the second OTP-ROM.

4. The method according to claim 3, wherein
the number of said first components is at least two, and at least one of the first components is connected to a first flash memory in which a startup program for self-starting the first component is stored.

5. The method according to claim 3, wherein
the number of said second components is at least one, and each second component comprises one second OTP-ROM.

6. The method according to claim 5, wherein the second component comprises at least one of the following:
a power module;
a user input module; and
a back end process module.

7. The method according to claim 1, wherein the method further comprises:
based on the first key matching the second key, performing matching success prompting; and/or
based on the first key not matching with the second key, writing the unique identifiers and the first key in a memory, and performing matching failure prompting.

8. The method according to any one of claim 1, wherein
the medical device comprises at least one of an ultrasound device, a monitor, an electrocardiogram anesthesia machine, a ventilator, an X-ray imaging device, and a magnetic resonance imaging device.

9. A medical device, comprising:
at least one first component, each first component storing a unique ID of said first component;
a first OTP-ROM, the first OTP-ROM storing a unique ID of the first OTP-ROM, and storing a second key;
at least one second component, each second component comprising a second OTP-ROM, and the second OTP-ROM storing a unique ID of said second OTP-ROM; and
a processor connected to the first component, the first OTP-ROM and the second component, the processor being configured to:
after the at least one first component, the first OTP-ROM, the at least one second component, and the processor are powered on, acquiring unique identifiers for each of the at least one first component, the first OTP-ROM, the at least one second component, and the processor;
calculating a first key according to the unique identifiers; and
matching the first key with a pre-stored second key, and determining whether the identity authentication is passed according to the matching result.

10. The medical device according to claim 9, further comprising:
a third component that is connected to the processor and connected to a second flash memory, the third component being configured to:
read a startup program from the second flash memory under the control of the processor; and
configure at least one of the first components using the startup program, so as to start at least one of the first components.

11. The medical device according to claim 9, further comprising:
a light emitting diode and/or a buzzer that is connected to the processor and configured to perform matching success and/or matching failure prompting under the control of the processor.

12. A non-transitory computer-readable medium, the non-transitory computer-readable medium having a computer program stored therein, the computer program having at least one code segment, and the at least one code segment being executable by a machine so as to cause the machine to perform operations of:
after all hardware to be authenticated of a medical device is powered on, acquiring unique identifiers of all the hardware to be authenticated;
calculating a first key according to the unique identifiers of all the hardware to be authenticated; and
matching the first key with a pre-stored second key, and determining whether the identity authentication is passed according to the matching result.

13. The non-transitory computer-readable medium according to claim 12, wherein determining whether the identity authentication is passed according to a matching result comprises:
based on the first key matching the second key, determining that the hardware identity authentication of the medical device is passed; and/or
based on the first key not matching the second key, determining that the hardware identity authentication of the medical device is not passed.

14. The non-transitory computer-readable medium according to claim 13, wherein all the hardware to be authenticated comprises:
a processor;
a first component;
a first one time programmable read-only memory (OTP-ROM) in which the second key is stored; and
a second component including a second OTP-ROM, and
wherein acquiring unique identifiers of all the hardware to be authenticated comprises:
acquiring a unique ID of the processor;
acquiring a unique ID of the first component; and
acquiring unique IDs of the first OTP-ROM and the second OTP-ROM.

15. The non-transitory computer-readable medium according to claim 14, wherein
the number of said first components is at least two, and at least one of the first components is connected to a first flash memory in which a startup program for self-starting the first component is stored.

16. The non-transitory computer-readable medium according to claim 14, wherein
the number of said second components is at least one, and each second component comprises one second OTP-ROM.

17. The non-transitory computer-readable medium according to claim 16, wherein the second component comprises at least one of the following:
a power module;
a user input module; and
a back end process module.

18. The non-transitory computer-readable medium according to claim 12, wherein the method further comprises:
based on the first key matching the second key, performing matching success prompting; and/or
based on the first key not matching with the second key, writing the unique identifiers and the first key in a memory, and performing matching failure prompting.

19. The non-transitory computer-readable medium according to claim 12, wherein
the medical device comprises at least one of an ultrasound device, a monitor, an electrocardiogram anesthesia machine, a ventilator, an X-ray imaging device, and a magnetic resonance imaging device.
